(19) United States
(12) Patent Application Publication
(10) Pub. No.: US 2019/0376968 A1
(43) Pub. Date: Dec. 12, 2019

(54) Medicament for Malignant Tumor Treatment

(71) Applicant: INTELLEXON GMBH, Pöcking (DE)

(72) Inventor: Wolfgang WÜRFEL, Sixtnitgern (DE)

(21) Appl. No.: 16/488,523

(22) PCT Filed: Feb. 22, 2018
(86) PCT No.: PCT/EP2018/054339

§ 371 Date: Aug. 23, 2019

(30) Foreign Application Priority Data
Feb. 27, 2017 (DE) 10 2017 001 875.8

(51) Int. Cl. G01N 33/569; C07K 16/28; G01N 33/50

(52) U.S. Cl. CPC G01N 33/56977; C07K 16/2818; C07K 2319/20; G01N 33/5011; C07K 2317/622; C07K 16/2809

(57) ABSTRACT

Methods for providing a medicament for the treatment of a malignant tumor taking into account the individually associated immune system, and also antibodies provided thereby in the use thereof, are disclosed, wherein the individual communication structure between the malignant tumor and the immune system is ascertained by determination of an expression pattern of embryonic HLA groups and by determination of a receptor type present on/in immunocompetent cells, and antibodies are provided of the type which specifically bond as ligands to the at least one determined receptor type and, as a result block or mask the receptor in such a manner that the at least one portion of the expression pattern cannot find their or can only bind there with lower action, but themselves do not inhibit the associated immunocompetent cell.

### FIELD OF THE INVENTION

The present invention relates for the area or providing medicaments for the treatment of malignant tumors.

### BACKGROUND OF THE INVENTION

In addition to the classic therapies for the treatment of malignant tumor diseases, e.g., resection, chemotherapy and radiation therapy, active or passive immunotherapies are increasingly used based on the administration of vaccines for initialing an immunological response or of antibody (fragments) for bonding to the malignant tumor. Medicaments for treating malignant tumors should be highly selective and not allow any resistances to be produced.

Nevertheless, tumor diseases have mechanisms for avoiding an immunological response. One such so-called escape mechanism is based on the MHC (Major Histocompatibility Complex), in particular with its HLA groups (Human Leucocyte Antigen), which serves for the cellular dialog in a human. In the literature, the abbreviation HLA can designate coding genes or proteins expressed by them. The concept HLA groups used in the following should designate here in particular the surface proteins expressed by the genes on the cell surface. In general, HLA groups can be divided, for example, into the following four classes:
(i) HLA groups A. B and C (MHC-I) which essentially identify all adult and somatic cells;
(ii) HLA groups D (DR, DP, DQ, etc.; MHC-II) which play an important part in immunocompetent cells and in the presentation of antigens;
(iii) HLA groups E, F and G which identify embryonic cells, in particular on the so-called invasion front;
(iv) HLA groups H and following ones, the so-called pseudogenes.

Malignant tumor cells can express typical embryonic HLA groups (i.e., HLA-E, HLA-F and/or HLA-G) on their surface. Embryonic HLA groups can contribute to the fact that malignant cells avoid the attack of the non-specific and/or specific immune defense of the organism itself. As a result of the expression of these typical HLA groups on the surface of the cells, the latter are rendered capable of activating corresponding receptors for immunocompetent cells. As a rule, receptors are involved which inhibit, after activation, the function of these immunocompetent cells, for example, the killer immunoglobulin-like receptors (KIR) on the natural killer cells or the leukocyte immunoglobulin-like receptors (LILR) on the lymphocytes.

Therefore, in particular the antigens HLA-E, F and G on the embryonic cells (especially on placental and trophoblastic ones) prevent the immune system of the mother from attacking the cells. In this manner, embryos can avoid the immunological response. This escape mechanism constitutes the backbone of the immunological control of pregnancy. A rejection reaction docs not take place and the genetically semi-foreign (father's foreign part 50%; or foreign embryo (in the case of single-cell donors or embryo donors or surrogate motherhood 100%) can be carried to full term.

Malignant tumors of very different tissues are capable of making use of this embryonic escape mechanism, wherein they suppress or avoid the immune defense. As a result, they are also capable of counteracting some therapeutic strategies, that is, to inhibit them to a strategy based on an attack. For this reason, it can be advantageous to include the immune system in order to treat the malignant tumor.

Production methods for antitumor medicaments are known from EP2 561 890 A1 in which, after the determination of an expression pattern of a malignant tumor cell, embryonic HLA groups expressed there are masked or destroyed. The masked or destroyed HLA groups can no longer inhibit the immune system so that an attack of the immunocompetent cells is to be reckoned with. For this, a masking of as many as possible malignant tumor cells is necessary. However, the masking of the HLA groups can also have effects on other, non-malignant tissues which also express embryonic HLA groups.

Given this background, the invention has the object of providing medicaments, antibodies for treating malignant tumors and the use of such antibodies.

### SUMMARY OF THE INVENTION

This object is solved by methods, antibodies and their use according to the independent claims. The dependent claims describe preferred embodiments.

A method according to the invention for providing a medicament for treating a malignant tumor with inclusion of the particular individually associated immune system comprises the determining of the individual communication structure between the malignant tumor and the immune system as well as the providing of antibodies.

In order to determine the individual communication structure between the malignant tumor and the immune system, at least one expression pattern of embryonic HLA groups present on the malignant tumor as well as at least one receptor status or receptor type present on/in immunocompetent cells of the immune system are determined. Even tumors or metastases which are classified as histopathologically identical can have expression patterns which are different inter-individually or intra-individually from one location to another location. Therapies, e.g., the administration of chemical therapeutic agents or hormonal antagonists can further influence the expression patterns. A determination of the individual expression patterns deals with these differences.

The determination of at least one receptor status, in particular of the receptor type, concerns at least one receptor type which is capable of bonding at least a part of the expression pattern as ligand and, based on this finding, of exerting and inhibitory action oil the immunocompetent cells. These receptor types of the particular receptor status are a class of surface proteins or transmembrane proteins which are expressed by the immunocompetent cells on their surface and initiate signal paths by signal transduction.

Inhibitory effect should designate here the immunomodulating effect which reduces or hinders the cytotoxic activity of the immunocompetent cells. This signal path can be initiated, for example, via the immunoreceptor, tyrosine-based inhibitory motive (ITIM), i.e., cytoplasmic phosphorylation.

The providing of antibodies refers to antibodies of the type which on the one hand specifically bonds as ligands to the at least one determine receptor type but docs not inhibit the associated immunocompetent cell and on the other hand blocks or masks the receptor in such a manner that the at least one part of the expression pattern cannot bond there or only with a slight effect. The masking or blocking of the receptor and therefore the preventing of the bonding of the expression pattern of embryonic HLA groups on this receptor can avoid their inhibitory effect.

In general, the antibodies provided in accordance with the invention are capable, by bonding to the receptor, of not exerting any inhibitory effect on the immunocompetent cells, i.e., they have no intrinsic activity. In contrast to the above, the expression patterns of embryonic HLA groups of the malignant tumor cell exert an inhibitory effect on the immunocompetent cells when bonding to the receptor.

In some embodiments the determining of the expression pattern comprises a check to see whether the embryonic HLA groups are preferably present or not. Furthermore, the determining of the expression pattern preferably comprises the quantitative determination of an expression level. For example, expression patterns or expression levels can be determined by known methods such as RNA sequencing. DNA microarrays. quantitative PCR (quantitative Polymerase Chain Reaction), expression profiling, SAGE (serial analysis of gene expression, serial gene expression analysis), etc.

The concept malignant tumor cell also comprises metastatic cells of the primary malignant tumor. The method according to the invention is carried out for preferably several, especially preferably for all metastases individually in order to examine any individual differences of the metastases, in particular their individual expression patterns of their embryonic HLA groups.

The at least one receptor type is preferably expressed as a transmembrane protein on the surface of the immunocompetent cells. In some embodiments the at least one receptor type comprises one or more of the following: KIR receptors (Killer Immunoglobulin-like Receptors), NKG2 receptors, LIL receptors (Leukocyte-Immunoglobulin-like Receptors).

Some NKG2 receptors, in particular KNG2 A, NKG2 B, NKG2 C, NKG2 D, NKG2 E and NKG2 F can bond, for example, HLA-E. Some LIL receptors, in particular LIL B1, LIL B2 and LIL B4, can bond, for example, HLA-F, KIR 2DL3 or LIL A2 can bond, for example. HLA-G.

The provided antibodies are of the type for bonding to at least one receptor type. Therefore, for example, the antibodies designated as anti-KIR 2DS1 bond as ligands to receptors of the type KIR 2DS1. The antibodies designated as anti-KIR 2DS4 can bond to receptors of the type KIR 2DS4.

In some embodiments the determining of the receptor type can also take into account which receptors were placed in the individual. The checking to see which receptors were placed in the individual can be carried out, for example, by gene analysis or expression analysis. If a certain receptor type is not present in the individual, an administration of antibodies against this resistor type is not indicated.

In some embodiments the immunocompetent cells comprise NK cells (Natural Killer cells) and/or lymphocytes.

In some embodiments the method furthermore comprises the providing of activating antibodies of the type which, after bonding to a receptor of the immunocompetent cells, initiate an activating action on the latter.

Furthermore, in some embodiments the method can comprise a determination of whether HLA-C is overexpressed. In the case of overexpression of HLA-C. Us expression pattern and a second receptor type for this expression pattern can be determined, namely, such a one which is capable of bonding at least one part of the HLA-C expression pattern as ligand and, based on this bond, to exert an activating effect on immunocompetent cells. For example, the second receptor type can be a receptor type from the group of KIR OS receptors which can exert an activating effect on NK cells. Examples of activating receptors which bond to HLA-C are KIR 2DS1. KIR 2DS2 or KIR 2DS4. Such receptors of the type KIR-DS can activate immunocompetent cells so that they produce, for example, substances which are growth-active or further the tumor growth such as cytokine or growth factors.

Alternatively, the second receptor type can be capable of bonding at least a pan of the HLA-C expression pattern as ligand and based on this bond, of exerting an inhibitory action of immunocompetent cells. Rot example, the second receptor type can be a receptor type from the group of KIR-DL receptors which can exert an inhibitory effect on NK cells. Examples of inhibitory receptors which bond to HLA-C are KIR 2DL1, KIR 2DL2, KIR 2DL3 or KIR 3DL3.

Starting from the second receptor type, second antibodies of the type can be provided which specifically bond to the second receptor type but do not inhibit or activate the associated immunocompetent cell and at the same time block or mask the second receptor in such a manner that the at least one part of the HLA-C expression pattern cannot bond there on only with a slight effect.

Furthermore, the invention provides antibodies which were made available by a method according to the invention; furthermore, the usage of antibodies according to the invention as medicament in the treatment of a malignant tumor is provided.

In some embodiments the method according to the invention comprises tire blocking or masking of receptors of the at least one determined receptor type on immunocompetent cells by the antibodies. In such a usage of the antibodies the bonding of embryonic HLA groups expressed on malignant tumor cells to the receptors of the at least one certain receptor type on the immunocompetent cells is prevented or reduced. The antibodies preferably display a high affinity to the receptor type, in particular comparable to or greater than or substantially greater than the affinity of the embryonic HLA groups of the malignant tumor cells. The affinity is preferably great enough to prevent a diffusing off and/or a competitive displacement of the antibodies.

In particular, the blocking or masking can take place in vivo, e.g., in the organism of the patient. In some embodiments the usage of the antibodies can take place locally or systemically. Local usage comprises, for example, the injection into the malignant tumor or into its vicinity. Systemic usage comprises, for example, the administration in one of the following ways: are orally, nasally, sublingually, rectally, subcutaneously, intravenously, percutaneously, etc.

Alternatively to a blockade in vivo, the blocking or masking can take place in vitro with subsequent transfusion of the immunocompetent cells, for example, in order to avoid systemic side effects. For a blockade in vitro, immunocompetent cells can be removed, exposed to the antibodies and transfused back after a masking has taken place.

In some embodiments of a use according to the invention the cells the expression pattern of which was determined and/or the immunocompetent cells the receptors of which are specifically bound by the antibodies stem from one and the same patient, for example, in order to avoid systemic side effects. Such embodiments can be preferred in particular if the inter-individual variation of the expression of embryonic HLA groups is great.

In some embodiments the immunocompetent cells the receptors of which are specifically bound by the antibodies stem from one donor. The donor can be a third person who is not sick from the malignant tumor. The immunocompetent cells can be injected, for example, after a blockade or masking of the receptors and are used to treat the malignant tumor, the expression of which was determined from embryonic HLA groups.

### BRIEF DESCRIPTION OF THE DRAWING

The attached drawings are referred to in the following description of exemplary embodiments:

FIG. 1 shows a flow chart of a method according to an exemplary embodiment.

FIG. 2 shows a schematic view of three malignant tumor cells of different individuals on whom a method according to an exemplary embodiment can be carried out.

FIG. 3 shows a schematic view of a malignant tumor cell and of an immunocompetent cell with a bond between an embryonic HLA group and a receptor of the immunocompetent cell,

FIG. 4 shows a schematic view of a malignant tumor cell and of an immunocompetent cell on which a method according to an exemplary embodiment can be carried out,

FIG. 5 shows a schematic view of a malignant tumor cell and of an immunocompetent cell on which a method according to an exemplary embodiment can be carried out.

FIG. 6 shows a schematic view of a malignant tumor cell and of two immunocompetent cells on which a method according to an exemplary embodiment can be carried out, and

FIG. 7 shows a schematic view of a malignant tumor cell and of an immunocompetent cell with masking of a receptor of the immunocompetent cell by an antibody according to an exemplary embodiment.

### DESCRIPTION OF PREFERRED EXEMPLARY EMBODIMENTS

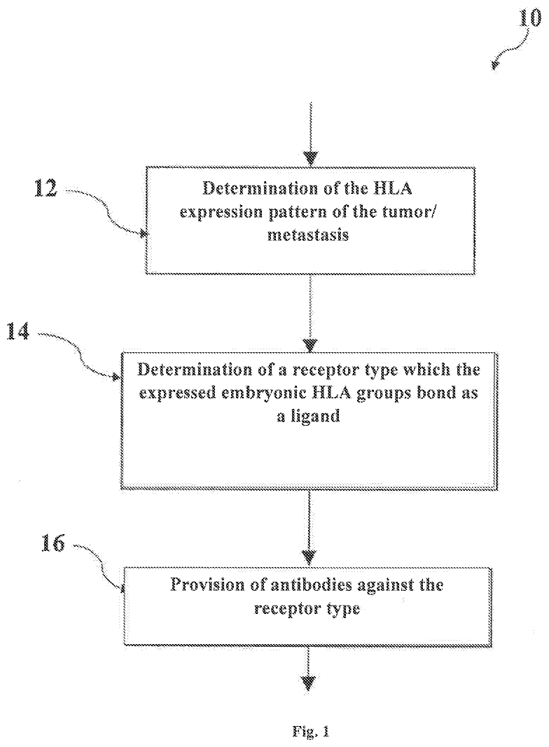

FIG. 1 shows a flowchart of a method 10 for providing a medicament. The method 30 comprises determining 12 of an expression pattern, determining 14 of at least one receptor type and providing 16 of antibodies.

The expressions of embryonic HLA groups located on the malignant tumor are determined during the determination 12 of (he expression pattern.

During determining 14 of at least one receptor type a receptor type is determined which is capable of bonding at least one part of the expression pattern of the embryonic HLA groups as ligand and, based on this bond, of exerting an inhibitory effect on the immunocompetent cells.

The antibodies provided in step 16 are of the type which specifically bond as ligands to the at least one certain receptor type of the immunocompetent cells and block or mask the receptor in such a manner that the at least one part of the expression pattern of the embryonic HLA groups cannot bond there or only bond with a lesser effect than the antibody but cannot itself inhibit the associated immunocompetent cells.

FIG. 2 shows a schematic view of three malignant tumor cells 20a, 20b, 20c of different individuals A, B and C on which a method according to the invention can be carried out. In particular, a particular expression pattern can be determined using each of the three malignant tumor cells 20a, 20b, 20c shown, in particular one for the particular individual or the individual expression pattern for the particular malignant tumor. The three malignant tumor cells 20a, 20b, 20c differ in their individual communication structure with the immune system.

A first malignant tumor cell 20a was taken from the individual A. This malignant tumor cell 20a may have been taken as part of a tissue sample of a malignant tumor of the individual A. The malignant tumor cell 20a comprises a plurality of membrane proteins (not shown) on its surface. A few of these membrane proteins belong to the embryonic HLA groups. The malignant tumor cell 20a comprises in particular embryonic HLA groups 24a of the HLA-E type and of the HLA-G type (but not of the HLA-F type). Therefore, an expression pattern 26a of the embryonic HLA groups 24a expressed by the malignant tumor cell 20a can be determined.

A second malignant tumor cell 20b which was taken from an individual B can coincide, partially deviate or completely deviate in the embryonic HLA groups 24b expressed by it from those expressed by the first malignant tumor cell 20a. In the case shown, the second malignant tumor cell 20b expresses embryonic HLA groups 24b of the type HLA-F. Therefore, an expression pattern 26b of the embryonic HLA groups 24b expressed by the malignant tumor cell 20b can also be determined.

An expression pattern 26c of embryonic HLA groups 24c which were expressed by the malignant tumor cell 20c can also be determined for a third malignant tumor cell 20c which was taken from an individual C. In the case shown, the expression pattern 26c comprises the embryonic HLA groups 24c of the type HLA-F and of the type HLA-G.

Starting from a certain expression pattern, at least one receptor type is determined which is capable of bonding as ligand one of the embryonic HLA groups contained in the expression pattern and, based on this bond, of exerting an inhibitory effect on immunocompetent cells.

When a receptor-ligand bond has been established, wherein the receptor was expressed on a first cell (e.g., an immunocompetent cell) and the ligand can be expressed on a second cell (e.g., a malignant tumor cell), a cellular dialogue takes place and this cooperation can develop, for example, an activating or inhibitory effect. Even the identification of cells is based on such receptor-ligand bonds.

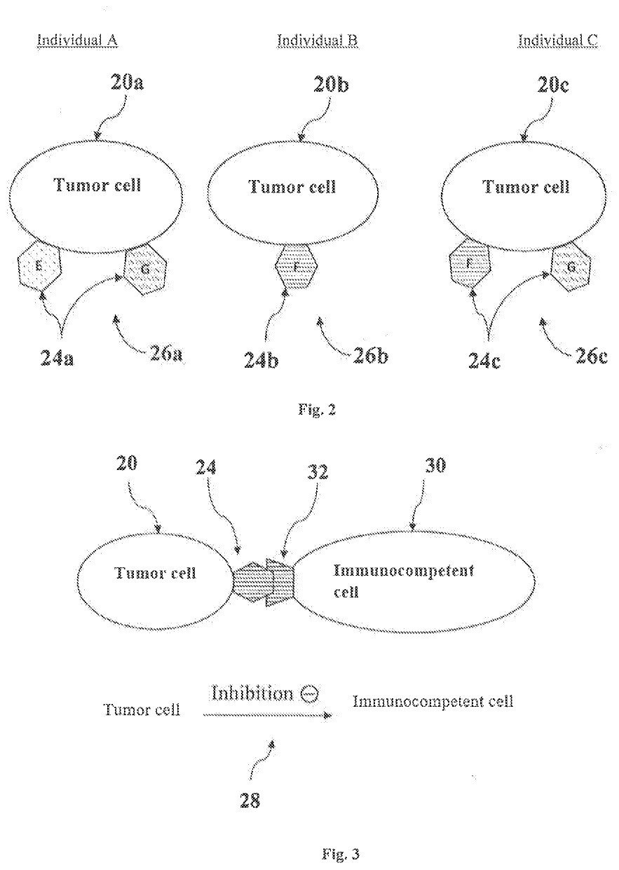

FIG. 3 shows a schematic view of a malignant tumor cell 20 and of an immunocompetent cell 30 with a bond between an embryonic HLA group 24 of the malignant tumor cell 20 and a receptor 32 of the immunocompetent cell 30. Based on the bond of the embryonic HLA group 24 shown, the malignant tumor cell 20 exerts an inhibitory effect 28 on the immunocompetent cell 30. This interaction between the expression pattern of the malignant tumor cell 20 and the receptor of the immunocompetent cell is characteristic for an individual communication structure between the malignant tumor and the immune system. This inhibitory effect has the consequence that an immune response of the immunocompetent cell 30 substantially does not take place.

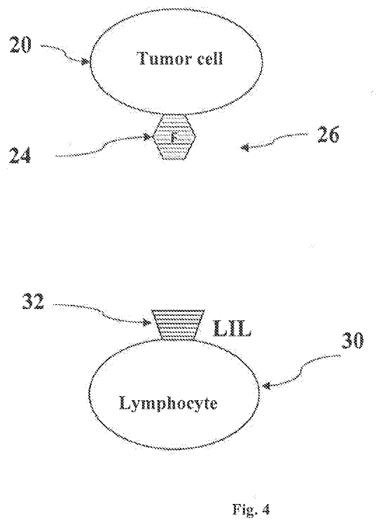

FIG. 4 shows a schematic view of a malignant tumor cell 20 and of an immunocompetent cell 30 on which a method according to the invention can be carried out. This exemplary embodiment illustrates in particular how at least one receptor type can be determined using a certain expression pattern. This determination contributes to the determining of the individual communication structure between the malignant tumor and the immune system.

Therefore, an expression pattern 26 of embryonic HLA groups can be determined on the malignant tumor cell 20 which pattern comprises the HLA groups of the type HLA-F.

Based on the expression pattern 26 determined, at least one receptor type 32 is determined which is capable of bonding as ligand one of the expressed embryonic HLA groups, namely, the HLA groups 24 of the HLA-F type and, based on the bond of the embryonic HLA group 24, of exerting an inhibitory effect on the immunocompetent cell 30. The immunocompetent cell 30 is a lymphocyte. A receptor type 32 which meets the above-cited prerequisites is LILR B1 (leukocyte immunoglobulin-like receptor B1).

Without carrying out the method according to the invention, the malignant tumor cell 20 might be able to bond by the HLA group 24 to the inhibitory receptor 32 of the lymphocyte 30 and therefore produce an inhibition of the immune system, this constitutes an escape mechanism of the malignant tumor cell in order to avoid the attack of the immune system.

An antibody can be provided in a method according to the invention based on the determined expression pattern 26 and the determined receptor type 32 which antibody suppresses this escape mechanism. The providing of the antibody can be carried out, for example, according to known production methods or isolating methods such as the hybridoma technology.

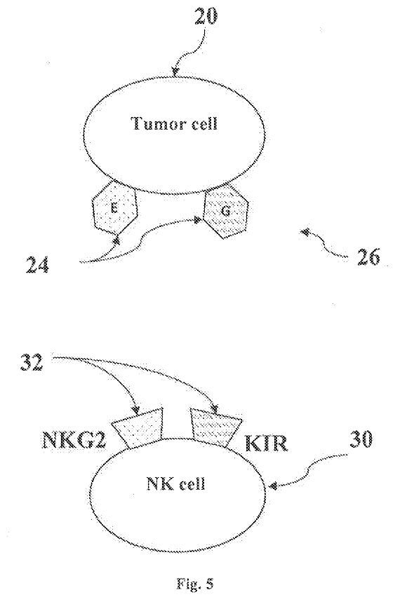

FIG. 5 shows a schematic view of another malignant tumor cell 20 and of a lymphocyte 30 on which a method for providing a medicament can be carried out. An expression pattern 26 can again be determined on the malignant tumor cell 20 by embryonic HLA groups expressed by the malignant tumor cell. The expression pattern 26 comprises HLA groups 24 of the types HLA-E and HLA-G. This exemplary embodiment illustrates in particular how several receptor types can be determined using a certain expression pattern which are characteristic for the individual communication structure between the malignant tumor and the immune system.

Two receptor types 32 are determined based on the expression pattern 26, namely, receptors NKG2 and KIR. These two receptor types 32 are capable of bonding one of the expressed, embryonic HLA groups 24 as ligand and, based on the bonding of the embryonic HLA group, of exerting an inhibitory effect on the immunocompetent cell 30. Therefore, the NKG2-receptors are capable of bonding HLA groups of the HLA-E type as ligand and, based on the bond of the HLA-E group, of exerting an inhibitory effect on the lymphocyte 30. Furthermore, the KIR receptors are capable of bonding HLA groups of the HLA-G type as ligand and based on the bonding of the HLA-G group, of exerting an inhibitory effect on the lymphocyte 30.

If, as in the example shown, several receptor types are determined, these several receptor types or at least a part of them can be blocked or masked, wherein a certain hierarchy of the blockade can be observed. Therefore, for example, the receptor of the KIR 2DL4 is considered as especially important for the inhibiting effect. Therefore, in order to avoid a side effect, a limitation to the most important receptor types can be preferred, especially in the case of a masking in vivo, in order to avoid systemic side effects.

In some exemplary embodiments individual receptor types can be blocked step by step, e.g., if a relative classification of the inhibiting effects of several receptor types is not yet known in the individual case. In the case of a lacking or insufficient immune response, one or more further or other receptor types can be blocked in further steps. Such a procedure can be preferred in particular in an in-vivo masking in order to avoid systemic side effects. In this manner a finely reduced control of the masking and the response of the immune system can be carried out.

In other embodiments all or at least a large number of receptor types can be simultaneously masked, e.g., if a relative classification of the inhibiting effects of several receptor types should not yet been known. Such a procedure can be preferred in particular in an in-vitro masking, among other things because systemic side effects from the antibodies are generally less probable there. In addition, in an in-vitro masking typically only a part of all immunocompetent cells is taken so that the influence of the masking on the immune system is limited in this respect.

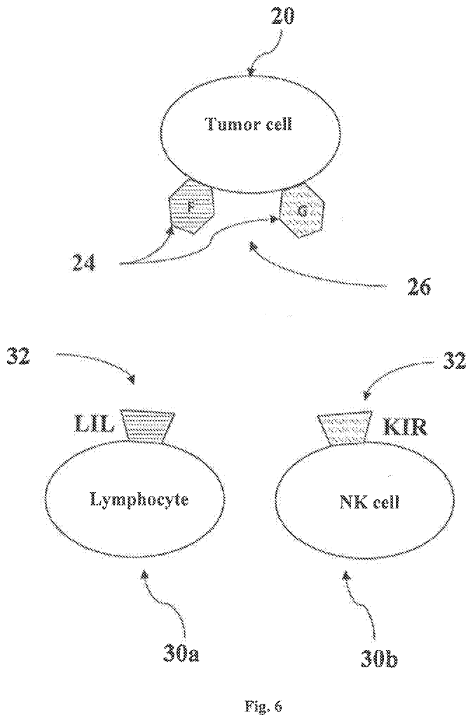

FIG. 6 shows a schematic view of a malignant tumor cell 20 and two immunocompetent cells, namely, a lymphocyte 30a and an NK cell 30b on which cells a method for providing a medicament can be carried out. Once again, an expression pattern 26 of embryonic HLA groups expressed by the malignant tumor cell can be determined on the malignant cell 20. The expression pattern 26 comprises HLA groups 24 of the type HLA-F and HLA-G. This exemplary embodiment illustrates in particular that the determined receptor types can have an inhibitory effect on different immunocompetent cells such as lymphocytes and NK cells. The individual communication structure between the malignant tumor and the immune system is determined in this manner.

Based on the two expressed HLA groups, two receptor types 32 are determined, namely, LIL-R (leucocyte immunoglobulin-like receptor) and KIR (killer cell immunoglobulin-like receptor). The LIL receptors are capable of bonding HLA groups of the HLA-F type as ligand and, based on the bonding of the HLA-F group, of exerting an inhibitory effect on the lymphocyte 30a. Furthermore, the KIR receptors are capable of bonding HLA groups of the HLA-G type as ligand and, based on the bonding of the HLA-G group, of exerting an inhibitory effect on the NK cells 30b. Therefore, the two receptor types 32 are capable of bonding one of the expressed embryonic HLA groups 24 as ligand and, based on the bonding of the embryonic HLA group, of exerting an inhibitory effect on different immunocompetent cells 30a, 30b.

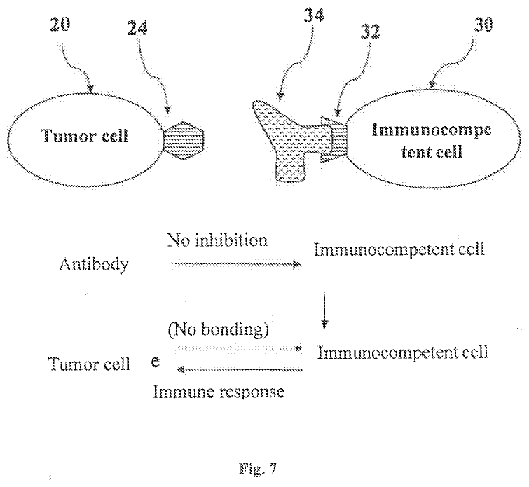

FIG. 7 shows a schematic view of a malignant tumor cell 20 with an HLA group 24 and an immunocompetent cell 30 with a receptor 32. Furthermore, an antibody 34 according to the invention is shown. This exemplary embodiment illustrates in particular how an antibody according to the invention can block or mask a receptor.

The receptor type 32 is basically capable of bonding the embryonic HLA group 24 of the malignant tumor cell 20 as ligand and, based on the bonding of the embryonic HLA group 24, of exerting an inhibitory effect on the immunocompetent cell 30. However, the receptor 32 of the immunocompetent cell 30 is masked by an antibody 34.

The antibody 34 is of the type which specifically bonds as ligand to the receptor 32 and as a result on the one hand blocks or masks the receptor 32 in such a manner that the embryonic HLA group 24 cannot bond to the receptor 32 or only with a slight effect, and on the other hand exerts no inhibitory effect on the associated immunocompetent cell. The principle of a ligand which bonds to a receptor and is not activated is known from pharmacology, for example, to the gonadotropin-releasing hormone (GnRH) receptors G used in reproduction medicine. GnRH antagonists are examples of ligands which bond to a receptor and do not activate it.

Without the antibody 34, the malignant tumor cell 20 could not bond by the HLA group 24 to the inhibitory receptor 32 of the immunocompetent cell 30 and in this manner produce an inhibiting of the immune system. This constitutes an escape mechanism of the malignant tumor cell for avoiding the immune system.

In the presence of the antibody 24 the inhibitory receptor 32 can be masked and the escape mechanism suppressed. The immune response of the immunocompetent cell 30 is not inhibited. Therefore, the antibody can serve for the treatment of the malignant tumor with the inclusion of the particular individually associated immune system.

The antibody used for a masking of the inhibitory receptor 32 can be included in a library of humanized antibodies against inhibitory receptor types of immunocompetent cells. That means that the variety of the library depends on the number of different inhibitory receptor types. If the bonding of another expression pattern, for example, of HLA pseudogenes, to these receptor types (the antibodies of which are already part of the library) are to be blocked or masked, then no expansion of the library (e.g., by producing new, humanized antibodies) is necessary but rather the existing library can be used for the providing of antibodies.

1. A method for preparing a medicament for the treatment of a malignant tumor with inclusion of the particular individual, associated immune system, comprising
(a) determining the individual communication structure between the malignant tumor and the immune system, comprising
determining at least one expression pattern of embryonic HLA groups present on the malignant tumor, and
determining at least one receptor type present on/in immunocompetent cells of the immune system which type is capable of bending at least a pad of the expression pattern as ligand and, based on this bond, of exerting an inhibitor)* effect on the immunocompetent cells, and
(b) providing antibodies of the type which
specifically bond as ligands to the at least one determined receptor type and as a result block or mask the receptor in such a manner that the at least one part of the expression pattern cannot bond there or can only bond there with a lesser effect, but do not inhibit the associated immunocompetent cell themselves.

2. The method according to claim 1, wherein the at least one receptor type comprises one or more of the following: KIR receptors, NKG2 receptors, LIL-R receptors.

3. The method according to one of the previous claims, additionally comprising:
the providing of activating antibodies of the type which initiate an activating effect on the immunocompetent cell after having bonded to a receptor of an immunocompetent cell.

4. The method according to one of the previous claims, comprising:
(a′) determining whether HLA-C is overexpressed and in the case of an overexpression of HLA-C
determining an HLA-C expression pattern of HLA-C groups,
determining a second receptor type which is capable of bonding at least a part of the HLA-C expression pattern as ligand and, based on this bond, of exerting an inhibitory or activating effect on immunocompetent cells, and
(b′) providing second antibodies of the type which
specifically bond to the second receptor type and as a result block or mask to the second receptor in such a manner that the at least one part of the HLA-C expression pattern cannot bond there or can only bond there with a lesser effect, but do not inhibit or activate the associated immunocompetent cell themselves.

5. Antibodies provided by a method according to one of the previous claims.

6. The use of antibodies according to claim 5 as medicament in the treatment of a malignant tumor.

7. The use according to claim 6, comprising: blocking or masking receptors of the al least one determined receptor type on immunocompetent cells by the antibodies.

8. The use according to claim 6 or 7, wherein
the immunocompetent cells, the receptors of which are specifically bonded by the antibodies, stem from a donor.
